(19) United States
(12) Patent Application Publication
(10) Pub. No.: US 2026/0108237 A1
(43) Pub. Date: Apr. 23, 2026

(54) DROP ON CARRIAGE ASSEMBLY, SYSTEM, AND METHOD

(71) Applicant: Innovative Medical Products, Inc., Plainville, CT (US)

(72) Inventor: Tamas KOVACS, Burlington, CT (US)

(73) Assignee: Innovative Medical Products, Inc., Plainville, CT (US)

(21) Appl. No.: 19/424,020

(22) Filed: Dec. 17, 2025

Related U.S. Application Data
(63) Continuation-in-part of application No. 18/448,408, filed on Aug. 11, 2023, now Pat. No. 12,508,013.
Application No. 18/448,408 is a continuation-in-part of application No. 18/341,856, filed on Jun. 27, 2023, now Pat. No. 12,440,199.
Application No. 18/341,856 is a continuation of application No. 17/364,689, filed on Jun. 30, 2021, now Pat. No. 11,717,280.

(51) Int. Cl. A61B 17/02

(52) U.S. Cl. CPC A61B 17/025; A61B 2017/0268; A61B 2560/0443

(57) ABSTRACT

The present invention relates to a carriage assembly and, more particularly, to a carriage apparatus, system, and method for coupling to a base plate and slidably holding a patient positioner, such as a distractor, at one or more angles with respect to an operating table. The carriage assembly may comprise a base, a jaw, and a fastener, which, when tightened, urges the jaw and base to clamp the track of a baseplate, the baseplate being coupled to a side rail clamp via pins. Alternatively, the carriage assembly may comprise a base and a lever assembly with open and locked positions, the latter securing the carriage to the track. Tapered openings in the carriage base may hold the patient positioner either vertically or at an angle with respect to the vertical. The carriages may be linearly translated to position the knee at a precise angles and provide enhanced structural stability.

### CROSS REFERENCE TO RELATED APPLICATIONS

This application is a continuation-in-part of, and claims priority to, co-pending U.S. patent application Ser. No. 18/448,408, filed on Aug. 11, 2023, entitled “Drop on Carriage Assembly, System, and Method”, which is a continuation-in-part of U.S. patent application Ser. No. 18/341,856, filed on Jun. 27, 2023, entitled “Modular Distractor Apparatus, System, and Method”, which granted on Oct. 14, 2025 as U.S. Pat. No. 12,440,199 B2, which is a continuation of U.S. patent application Ser. No. 17/364,689, filed on Jun. 30, 2021, entitled “Modular Distractor Apparatus, System, and Method”, which granted on Aug. 8, 2023 as U.S. Pat. No. 11,717,280 B2, which are herein incorporated by reference in their entirety.

### FIELD OF THE INVENTION

The present invention relates generally to patient positioning systems for surgical applications, and more particularly, to a carriage apparatus, system, and method for coupling to a base plate capable of slidably translating therealong, the carriage configured to hold a limb positioner or distractor at a desired position relative to an operating table.

### BACKGROUND OF THE INVENTION

Surgical procedures increasingly require precise and predictable positioning of the joint, limb, or other body part corresponding to the surgical site. Optimal positioning requires a large range of easily available positive positioning adjustments, so that the limb, joint, or other body part can be precisely positioned and thereafter maintained in that desired position. First, specialized surgical procedure operating room (OR) or other support tables have been developed for precision positioning of the body part, but these specialized OR tables are generally useful for a limited type of surgeries, are expensive, and take up a lot of room. It would be desirable to have a patient positioning system, such as for knee replacement surgeries, that couples to a standard OR table with side rail. Second, it would be desirable to have a patient positioning systems that couple to a standard OR table with side rail wherein the system does not obstruct access by the surgeon to the surgical site. Third, it is desirable to have a patient positioning device that maintains a desired position throughout the surgery, without slipping, loosening, or otherwise moving during the procedure. Fourth, it is desirable to have a patient positioning device that facilitates single-handed operation of the device by the surgeon and/or surgical support staff. Among other reasons, devices supporting single-handed operation frees up the other hand of the operator to allow for simultaneous tasks, such as supporting the patient limb and the like. Single-handed operation advantageously provides the surgeon with great flexibility, and the like, during the course of the operation, where adjustments and/or set up of the positioning device(s) may be desired. Fifth, it is desirable to have a patient positioning device that eliminates parts and complexity thereof. Sixth, it is desirable to have a patient positioning device that is easily sterilizable between surgeries.

Consequently, there is a need for a patient positioning system that couples to OR tables having standard side rails, that is out of the surgeon's way, that promotes ease of use, and that has improved holding characteristics.

### SUMMARY OF THE INVENTION

The present invention advantageously fills the aforementioned deficiencies by providing a carriage capable of slidably coupling a patient positioning device, such as a distractor, to a base plate for use in knee replacement surgeries and other surgical procedures.

It is an object of the present invention to provide a carriage configured to couple a patient positioning device to a base plate, wherein the carriage, patient positioning device, etc., is capable of coupling to a standard OR table, such as an OR table including a side rail having industry-standard cross-sectional dimensions.

It is an object of the present invention to provide a carriage configured to couple a patient positioning device to a base plate, wherein the carriage, patient positioning device, etc., is positioned underneath or substantially underneath, the patient and/or surgical site, thereby reducing obstruction of the system and providing greater access for the surgeon and/or operating room personnel.

It is also an object of the present invention to provide a patient positioning boot and/or boot carriage that is mechanically decoupled from a distractor, wherein, for example, the carriage couples the distractor to the base plate, the distractor being located at a first position of a track of the base plate, and the patient positioning boot is coupled to a different carriage, the boot being located at a second position of the track. In this example, the carriage according to the present invention reflects an independent anchoring point, which may advantageously provide additional support, rigidity, and/or enhanced holding of the patient throughout the surgical procedure. Such support may be favorable for surgical applications requiring or benefiting from a high degree of stability, such as robotic surgical applications. Such a system may effectively form a triangle shape, a shape which is commonly known to enhance structural stability, the side of the triangle being formed by (i) a baseplate coupled to the OR table surface, (ii) a distractor disposed upwardly from the OR table surface from a dedicated carriage, and (iii) the lower leg of a patient, i.e., tibia/fibula, wherein the patient foot is coupled to a boot, the boot being coupled to the baseplate via a separate dedicated carriage.

It is also an object of the present invention to provide a drop-on carriage, i.e., a carriage capable of connecting to a baseplate at a location between the ends of the track, such as along the length of the baseplate track. As compared to a clamp and/or carriage that is only capable of coupled to a track of a baseplate from either end, the drop on carriage advantageously can be added and/or removed from the surgical patient positioning system as desired by the surgeon. In one aspect, removal of the distractor and a corresponding low-profile, drop-on carriage from the patient positioning system, allows the surgeon to test the knee replacement, wherein the leg is full extended and laid flat on the table and/or baseplate or track. It may then be desirable to reestablish the initial surgical position with the knee being bent in the aforementioned triangle position, for further surgical correction of the knee replacement.

It is also an object of the present invention to provide a carriage configured to couple to a support platform or base plate, wherein the base plate may be clamped to the side rail of an operating table. In one or more embodiments, the carriage may be configured to hold a distractor apparatus for supporting and distracting the knee joint. Additional components may be coupled to the base plate and disposed proximate the carriage along another portion of the track of the baseplate, such as a boot, which may also be clamped to and/or translated linearly along the length of the base plate. As another example, one or more retractor clamps may be clamped to, and/or translated along, the base plate, wherein the retractor clamp may be configured to support a retractor system. Advantageously, the carriage according to the present invention may be shaped having a low profile, or otherwise shaped to be employed with any additional components, such that during repositioning, all components may move in concert, allowing for small adjustments to the angle of the knee changes. This may facilitate ‘free flow’ of the surgical procedure from one step to the next, with minimal time in between to reduce the total amount of time required, increase accuracy of the surgical procedure, and/or increase effectiveness of the patient positioning system.

Other desirable features and characteristics including will become apparent from the subsequent detailed description, the drawings, and the appended claims, when considered in view of this summary.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present disclosure, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 illustrates a carriage slidably coupled to a base plate, according to a first embodiment of the present invention;

FIG. 2 illustrates a top, front, right-side perspective view of an assembled carriage, according to the first embodiment of the present invention;

FIG. 3 illustrates an exploded, top, front, right-side perspective view of a carriage, according to the first embodiment of the present invention;

FIG. 4 illustrates a carriage slidably coupled to a base plate, according to a second embodiment of the present invention;

FIG. 5A illustrates a top, front, right-side perspective view of a carriage in an open position, according to the second embodiment of the present invention;

FIG. 5B illustrates a top, front, right-side perspective view of a carriage in a locked position, according to the second embodiment of the present invention;

FIG. 6 illustrates an exploded, top, front, right-side perspective view of a carriage, according to the second embodiment of the present invention.

### DETAILED DESCRIPTION

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the present invention. For a better understanding of the present invention, reference will be made to this Detailed Description, which is to be read in association with the accompanying drawings, claims, and abstract, which are incorporated in and constitute a part of this specification, show certain aspects of the subject matter disclosed herein and, together with the description, help explain some of the principles associated with the disclosed implementations.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

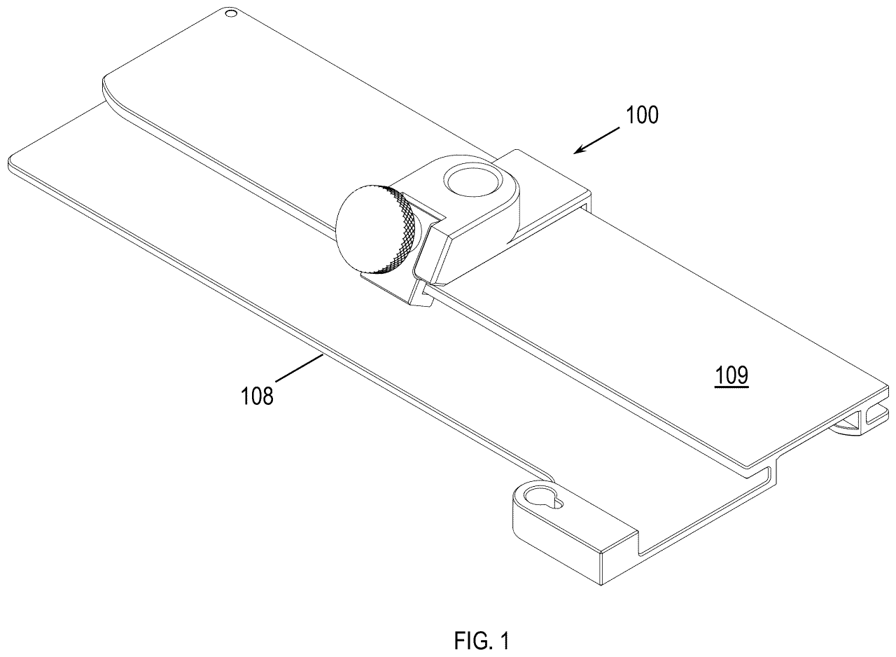

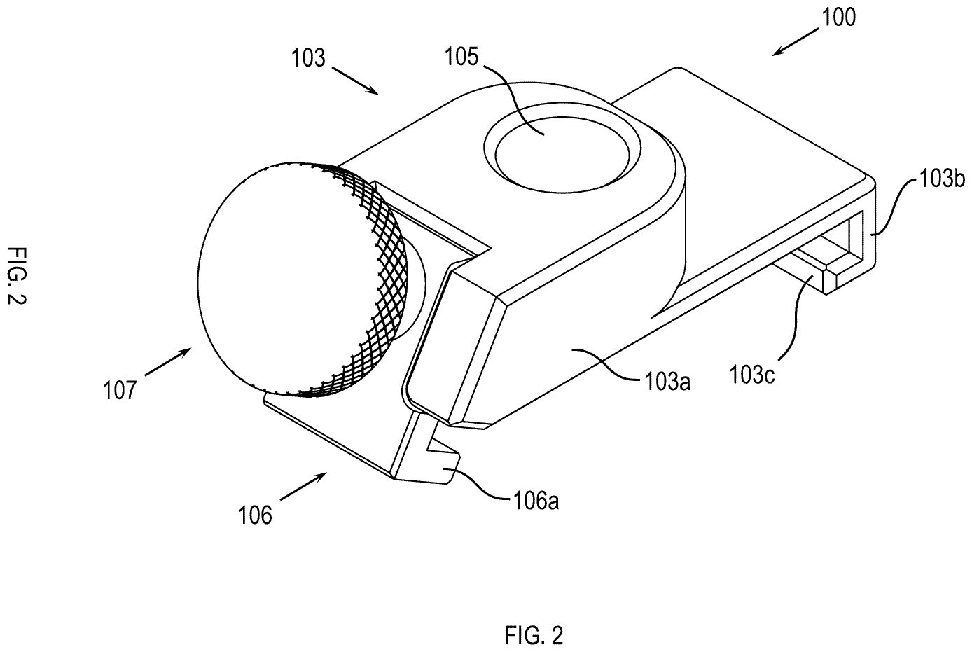

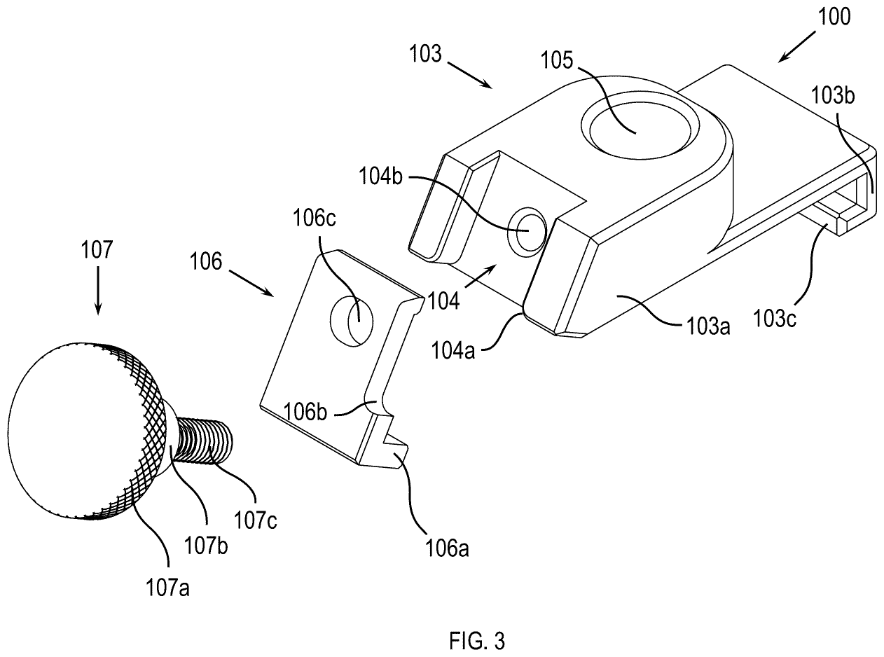

FIGS. 1-6 illustrate certain embodiments and features generally shown as carriage apparatus, system, and method 100, according to the present invention. The carriage 100 may be employed as a component of a patient positioning system for surgical applications; however, use of the carriage 100 is nonlimiting. Carriage 100 may be useful for a variety of applications, such as in the manufacturing industry, and other fields of use. For example, carriage 100 may be employed to secure and hold a work tablet or electronic device to a structure, for ease of use and display while in the field. Therefore, the applications of the carriage 100 shall be considered nonlimiting.

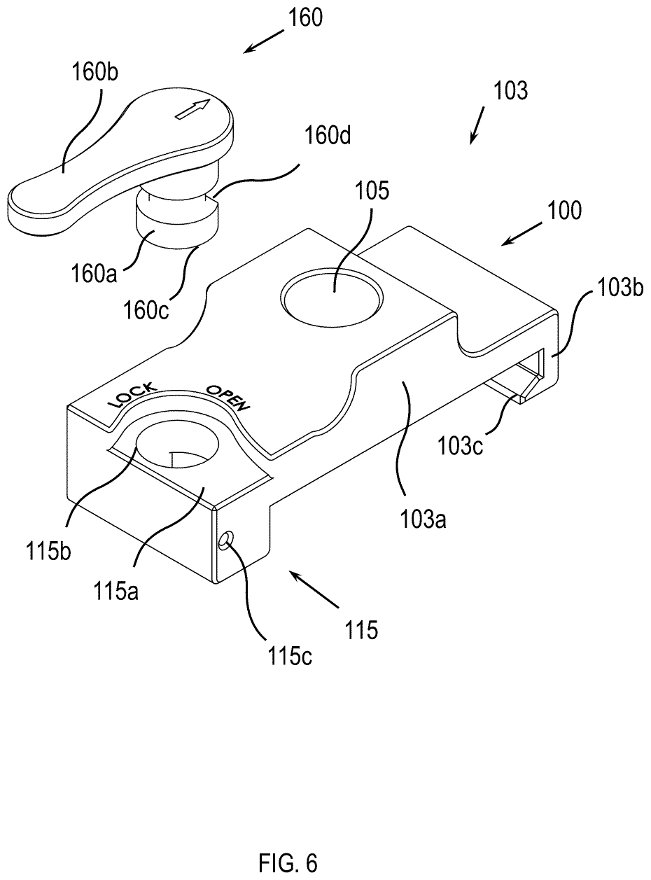

According to the present invention, carriage 100 may be generally adapted to couple a distractor to a baseplate 108, which may be useful for knee replacement surgeries, or other surgeries, as shown in FIGS. 6 and 8 of U.S. patent application Ser. No. 18/448,408 herein incorporated by reference in its entirety. Baseplate 108 may be a separable baseplate 108, such as that disclosed by the applicant in U.S. Pat. No. 11,839,574 A1, entitled “Split Base Plate Assembly Using Pins,” which is also incorporated herein by reference in its entirety. Such a baseplate system may be used to provide a track portion 109 extending outwardly and/or upwardly from an operating room (OR) table or other support 101, and adapted for receiving and coupling to carriage 100. The carriage 100 may then advantageously be dropped on and coupled to the base plate 108 along any portion of the track 109 and, for example, translated therealong to a desired position. Placement and/or assembly of the carriage 100 may be done via single-handed operation. The carriage 100 may be further configured to hold a patient positioner for a limb, such as a distractor adapted to receive, hold, and/or distract a joint of, a lower limb of a patient.

FIGS. 1-3 illustrate certain views of a first embodiment of the carriage 100, according to the present invention. The carriage 100 may comprise a carriage body 103, a jaw 106, and a fastener assembly 107. The carriage body 103 may include a base 103a, a receiver portion 103b, a flange portion 103c, a recess 104, and an opening 105. The receiver portion 103b extends outwardly from the base 103a, and, in coordination, the receiver portion 103b, the flange portion 103c, the underside of the carriage body 103, and a jaw 106 provide a C-shaped clamping assembly to fixedly couple the carriage 100 to the baseplate 108 via the track 109. A recess 104 formed in the carriage body 103 may include a chamfer 104a and an opening 104b. The recess 104 may be generally offset from the surface formed by the side of base 103a, or otherwise configured to receive the jaw 106, such that the jaw 106 is at least somewhat limited in terms of rotational movement, but while preferably also allowing for slight angular adjustment of the jaw 106 relative to the carriage body 103. In this manner, slight angular adjustments of jaw 106 can facilitate more secured holding of the carriage 100 to the track 109 in instances where, for example, the track is slightly angled relative to the position of the carriage 100 on the track 109. The opening 105 may be configured to receive a tapered end of an adapter of distractor assembly, or other positioner, assembly, etc. Recess 104 may comprise a recess chamfer 104a and a recess opening 104b. The recess chamfer 104a may be chamfered or filleted, and the recess opening 104b may be formed into the base 103a and oriented orthonormal relative to the surface formed by the recess 104.

In a preferred embodiment, the opening 105 may be disposed normal to the surface of the base plate 108 when the carriage 100 and base plate 108 are in an assembled configuration or, alternatively, opening 105 may be disposed at an acute angle relative to the surface normal of the base plate 108. The base 103a of carriage body 103 may have an upper surface that is parallel to the surface formed by the upper surface of track 109, such as shown in FIG. 1. The base 103a and other components may be made in other shapes or forms than that shown, but the orientation of the opening 105 relative to the support table 101 is of a primary concern, because that orientation establishes the positioning of the patient via the components extending outwardly from the opening 105, such as the positioning of the distractor and/or patient positioning pad described in U.S. patent application Ser. No. 18/448,408, which are suitable for coupling and supporting the patient relative to the support table 101.

As shown in FIGS. 1-3, concerning the first embodiment of the carriage 100 according to the present invention, the jaw 106 may comprise a jaw flange 106a, a jaw key 106b, and a jaw slot 106c. The jaw 106 may operably couple to the base 103 of the carriage body 103a and secured thereto via operation of the fastener assembly 107, which may take the form of a threaded knurled knob or other known fastening method and/or arrangement. The jaw flange 106a may be configured to engage a side of the track 109 and a jaw key 106b formed in a complementary shape to the recess chamfer 104a, thereby configured to facilitate only proper mating between the jaw 106 and the slot 104, by limiting the extent to which the jaw 106 can rotate relative to the base 103a in the manner previously described. The fastener assembly 107 of the carriage 100 may include a knurled portion 107a, a neck portion 107b, and a threaded portion 107c. In operation, the knurled portion 107a may be manually rotated to advance the threaded portion 107c into the opening 104b. Without complete advancement of the fastener assembly 107 into the recess opening 104b, the jaw 106 may be moveable, allowing for translation of the carriage 100 along the track 109. Upon clockwise turning of the fastener assembly 107, the jaw 106 may be loosely urged toward the base 103a, and upon subsequent tightening of the fastener assembly 107, the neck 107b and slot 104 may rigidly position the jaw 106 around the rail 109 of the base plate 108 to achieve effective clamping. This arrangement facilitates single-handed operation of the carriage 100.

Because surgical equipment requires sterilization prior to use in surgical applications, the carriage 100 is configured for disassembly such that small gaps and/or volumes are reduced or eliminated. Pockets, blind openings, reentrant surfaces and the like may retain non-sterile matter that may contaminate the surgical environment. Therefore, when the carriage 100 is disassembled prior to sterilization, and then reassembled prior to use, it may be advantageous to have mechanical datums that prevent improper assembly. The jaw 106, in particular, may be subject to upside-down or reverse assembly. To prevent mis-assembly, one or more datums 106b may be disposed in the jaw 106 and/or slot 104 of the base 103a. In addition, the threaded opening 104b within the slot 104 may be offset from the centerline of the base 103a. That way, if improperly oriented, the holes in the base 104b and jaw 106c may not line up. Alternatively, other matching keys may be disposed in the slot 104 and jaw 106, without limitation, such that proper assembly may be ensured. In another aspect, certain openings within base 103a may be configured as through-holes. For example, tapered opening 105, and slot opening 104b may be configured as through holes. Alternatively, such openings may be configured as blind holes.

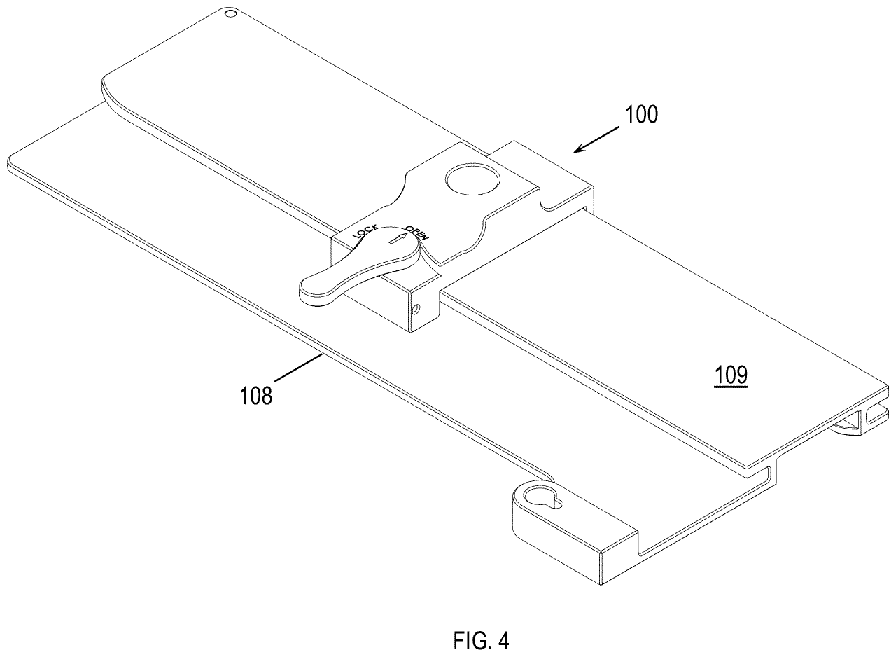

FIGS. 4-6 illustrate a second embodiment of the carriage 100 according to the present invention. Carriage 100 of this embodiment may comprise a carriage body 103 and a lever assembly 160. The carriage body 103 may comprise a base 103a, a receiver portion 103b including a flange portion 103c, an opening 105, and a lever receiver portion 115. The receiver portion 103b extends generally outwardly from the base 103a, and, in coordination, the receiver portion 103b, the flange portion 103c, the underside of the carriage body 103, and the lever assembly 160 provide a C-shaped clamping assembly to fixedly couple the carriage 100 to the base plate 108 via the track 109. The opening 105 of the carriage body 103 may be configured to receive a tapered end of an adapter of a distractor assembly, as described above with respect to the first embodiment; opening 105 may alternatively be configured to receive other positioners, assemblies, and the like. The opening 105 may be disposed normal to the surface of the base plate 108, or at an acute angle relative to the surface normal of the support table, also as described above with respect to the first embodiment. As shown in FIG. 6, the lever receiver portion 115 may comprise a lever receiver recess 115a, a lever receiver opening 115b, and a pin screw opening 115c.

The lever assembly 160 of carriage 100 may comprise a cam 160a and a handle 160b. Cam 160a may comprise a circular portion 160c and a flat portion 160d, which work in conjunction with the carriage body 103 to operably and/or fixedly couple the carriage 100 to the track 109. The cam 160a may be configured to be received through the lever receiver opening 115b of lever receiver portion 115, wherein the lever receiver opening 115b is exposed along the bottom side of base 103 to track 109 (when in an assembled configuration thereto). A pin screw (not shown) may be inserted into pin screw opening 115c to inhibit removal of the lever assembly 160 when in use. The lever assembly 160 may be rotated between an open position 500 and a locked position 501, thereby enabling operably coupling of the carriage 100 to the track 109.

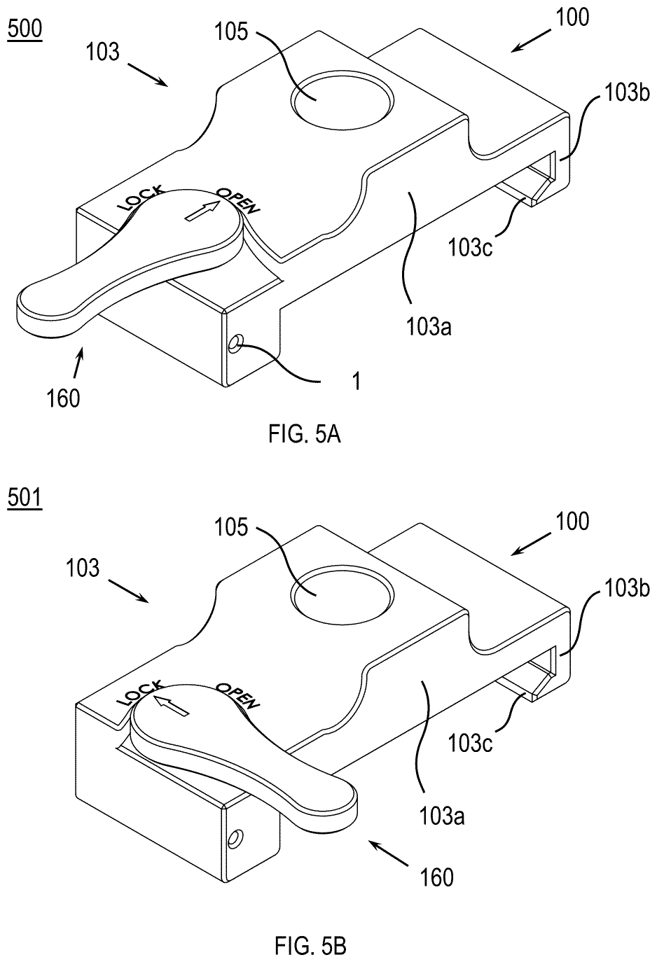

FIGS. 5A and 5B illustrate the second embodiment of clamp 100 in open and locked positions, 500, 501, respectively, according to the present invention. The desired position 500, 501, may be selected by the operator to position the lever assembly 160. In the open position 500, illustrated in FIG. 5A, the lever provides clearance between the clamp 100 and the track 109, thereby allowing drop-on assembly thereto and translation therealong. In the closed position, illustrated in FIG. 5B, the lever provides mechanical communication between the carriage 100 and track 109. Referring to FIG. 6, the mechanical clearance or engagement is provided by a cam 160a disposed on the lever assembly 160. In the locked position, the cam 160a interferes with the track 109 with sufficient friction so as to fixedly couple the carriage 100 to the track 109. The lever is advantageously configured for quick, single-handed operation.

Regarding sterilizability, FIG. 6 illustrates the open design of the second embodiment of carriage 100, which is devoid of small gaps and/or volumes, and which predominantly comprises surfaces with line-of-sight orientation relative to the exterior (minimal reentrant surfaces), which facilitates sterilization thereof. The simplified design advantageously features a reduced number of parts which may improve the ease of disassembly for sterilization and assembly for use.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A carriage assembly for securely coupling a surgical joint distractor to a track of a baseplate, the baseplate being securely coupled to a support table, the carriage assembly comprising:
a carriage body including a base, a receiver portion extending outwardly from said base, a flange disposed at an outward end of said receiver portion, and a lever receiver portion,
wherein the carriage body is adapted to receive the track in coordination with the receiver portion and flange, and wherein the lever receiver portion includes a lever receiver opening; and
a lever assembly including a cam and a handle, wherein the cam is configured to be received in the lever receiver opening;
wherein, in an assembled configuration, with the handle in an open position, the carriage assembly is configured to slide along the track, and with the handle in a locked position, the carriage assembly is fixedly coupled to the track.

2. The carriage assembly of claim 1, wherein the carriage body further comprises one or more openings, each opening being configured to receive the surgical joint distractor.

3. The carriage assembly of claim 2, wherein one opening of said one or more openings is configured to orient the surgical joint distractor, either orthonormal to a surface defining the support table, or at an angle from orthonormal ranging from about 5 degrees and about 30 degrees.

4. The carriage assembly of claim 3, wherein said one opening of said one or more openings is configured to orient the surgical joint distractor at an angle from orthonormal of 15 degrees.

5. The carriage assembly of claim 1, wherein the cam of the lever assembly is tapered to provide for a proportional increase in tightening of the lever assembly and carriage body to the track of the baseplate.

6. The carriage assembly of claim 1, wherein the lever receiver portion further comprises a pin screw opening adapted to receive a pin screw, such that when in the assembled configuration the lever assembly remains operably coupled in both the open and locked positions.

7. A carriage assembly for securely coupling a device to a track, the carriage assembly comprising:
a carriage body including a base, a receiver portion extending outwardly from said base, a flange disposed at an outward end of said receiver portion, and a lever receiver portion,
wherein the carriage body is adapted to receive the track in coordination with the receiver portion and flange, and wherein the lever receiver portion includes a lever receiver opening; and
a lever assembly including a cam and a handle, wherein the cam is configured to be received in the lever receiver opening;
wherein, in an assembled configuration, with the handle in an open position, the carriage assembly is configured to slide along the track, and with the handle in a locked position, the carriage assembly is fixedly coupled to the track.
